(19) United States
(12) Patent Application Publication
(10) Pub. No.: US 2007/0098659 A1
(43) Pub. Date: May 3, 2007

(54) Method for making a cosmetic device having discrete elements

(76) Inventors: Raymond Ip, Plainsboro, NJ (US); Robert A. Brennan JR., Mercerville, NJ (US); Michael W. Eknoian, Warren, NJ (US); John F. Poccia III, Monmouth Beach, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/263,168

(22) Filed: Oct. 31, 2005

(51) Int. Cl. A61K 8/73

(52) U.S. Cl. 424/70.11; 424/70.13; 424/70.17

(57) ABSTRACT

The present invention relates to a method of making a cosmetic device comprising a cosmetic composition and a plurality of discrete elements using a releasably connected matrix of discrete elements.

### BACKGROUND OF THE INVENTION

Cosmetic bars have been used over the years to deliver many benefits to the skin. Cosmetic bars containing ingredients such as cocoa butter have been used to moisturize or protect the skin. More conventionally, bars containing soap and moisturizers have been used to cleanse and condition the skin. The bars have several problems associated with them. In particular, they are very slippery when wet. Cleansing bars tend to slip out of the consumer's hand during use in the shower or bath. The consumer then has to bend over or kneel down to pick up the cleansing bar from the floor of the shower.

Additionally, it is somewhat awkward to apply soap with a washcloth or sponge since it involves the use of two separable articles, one being extremely slippery when wet and tending to slide from the user's hands quite easily. Wrapping the washcloth around the soap may be a temporary solution but it is not completely satisfactory. Similarly, making a pouch in the sponge to contain the bar of soap leaves the sponge permanently saturated with the soap and slimy after its initial use. Sewing a bar of soap between two plies of washcloth likewise produces an article that is permanently slimy after use.

Others have tried to extend the life of a cosmetic or cleansing bar that is typically fragile when reduced to a sliver. The sliver will often break or become hand to handle. Solutions to these problems may include the incorporation of hair, sponges, fibers, etc. Examples of such disclosures are described in U.S. Pat. Nos. 681,324; 389,296; 488,393; and 5,221,506.

Skin cleansing compositions having abrasive particles incorporated as scrubbing aids are known in the art. For example, LOOFAH Exfoliating Soap is a commercially available soap bar from Earth Therapeutics. The soap bar has small particles of a chopped up loofah or puff dispersed throughout.

U.S. Pat. No. 6,818,603 describes a cleansing bar comprising (a) a cleansing composition; and (b) a plurality of discrete elements having a length to diameter ratio of from about 50 to 1 to about 1,000,000 to 1. The discrete elements may be fibers, filaments, particles, and mixtures thereof. In addition, the discrete elements may comprise monocomponent or multicomponent elements, including core-sheath structures. Such a device is useful for exfoliating the skin while cleansing. However, the dispersion of discrete elements within a cosmetic composition, particularly in a specific configuration, often poses a technical challenge. Factors such as the densities of the discrete elements and the cosmetic composition, the viscosity of the cosmetic composition, and the fragility of the discrete elements complicate the task. The discrete elements may aggregate for example by sinking to the bottom or floating to the top of the cosmetic composition, or become damaged during mechanical processes such as stirring. Conversely, the discrete elements may damage the equipment used to make the cosmetic device. Determination of the correct amount of discrete elements for the cosmetic device can also be difficult.

Accordingly, a need exists for a method of producing a cosmetic device comprising discrete elements that provides improved control of the configuration of the discrete elements within the cosmetic device. The present invention provides such a method, employing a matrix made of releasably connected discrete elements. The matrix is contacted with a flowable cosmetic composition, whereupon the discrete elements are allowed to separate.

### SUMMARY OF THE INVENTION

The invention provides a method of making a cosmetic device comprising a cosmetic composition comprising a plurality of discrete elements, said method comprising the steps of: a) contacting a releasably connected matrix of discrete elements with a flowable cosmetic composition, such that the cosmetic composition saturates at least a portion of the matrix; and b) allowing the discrete elements to separate within the cosmetic composition. The method may further comprise molding the cosmetic composition containing the discrete elements and/or allowing the cosmetic composition containing the separated discrete elements to form a solid or semi-solid, for example by cooling.

### DETAILED DESCRIPTION OF THE INVENTION

As used herein the term “cosmetic” shall include conditioning, moisturizing, cleansing, or any other treatment that is applicable to the skin of the human body. Accordingly, the cosmetic device may be used to deliver any such treatment to the skin.

The cosmetic device comprises a cosmetic composition and discrete elements. The finished cosmetic device may be a liquid, solid or semi-solid (including gel).

Cosmetic Composition

The cosmetic composition may be selected for example from moisturizing compositions, cleansing compositions, or any composition that may provide a benefit to the skin.

In one embodiment, the cosmetic composition is a cleansing composition. Suitable cleansing compositions are liquid, solid or semi-solid at room temperature. Examples of useful cleansing compositions include, but are not limited to, fatty acid soaps, including glycerin soaps, synthetic detergents and mixtures thereof. Solid cleansing compositions are extensively taught in Soap Technology for the 1990's, the contents of which are incorporated herein by reference. It is desirable that the cleansing composition be flowable.

In one embodiment of the invention, the cleansing composition comprises glycerin soap. Examples of glycerin soaps useful in the present invention include but are not limited to those disclosed in U.S. Pat. Nos. 4,405,492 and 4,879,063, the disclosures of which are hereby incorporated by reference.

Examples of suitable fatty acid soaps include soaps derived from hydrocarbon chain lengths of from approximately 10 to 22 (including carboxyl carbon) and may be saturated or unsaturated. The soap may be, for example, the sodium salt, potassium salt, ammonium salt, triethanolammonium salt and mixtures thereof.

Suitable synthetic detergents include those known in the art for the desired purpose. Examples of detergents useful for personal cleansing include the isethionates, sarcosinates, and glyceryl ether sulfonates which may be pure chain length variants or those derived from commercial oils such as coconut oil.

Numerous other detergents are appropriate for this invention. These include anionic acyl sarcosinates, methyl acyl taurates, N-acyl glutamates, alkyl sulfosuccinates, alkyl phosphate esters, ethoxylated alkyl phosphate esters, trideceth sulfates, protein condensates, mixtures of ethoxylated alkyl sulfates and alkyl amine oxides, betaines, sultaines and mixtures thereof. Included are the alkyl ether sulfates with 1 to 12 ethoxy groups, especially ammonium and sodium lauryl ether sulfates. Alkyl chains for these other detergents are C₈-C₂₂, preferably C₁₀-C₁₈. Alkyl glucosides and methyl glucoside esters are preferred mild nonionics, which may be mixed with other mild anionic or amphoteric surfactants in the compositions of this invention.

In one embodiment, the cleansing composition may comprise the following ingredients:

| | % w/w |
|---|---|
| Propylene Glycol | 32.6 |
| Glycerin | 12.6 |
| Sodium Hydroxide Solution (50%) | 8.6 |
| Stearic Acid | 10.1 |
| Myristic Acid | 10.1 |
| Lauric Acid | 8.1 |
| Water (H2O) | 4.0 |
| Sodium Lauryl Sulfate | 4.0 |
| Lauryl Methyl Gluceth-10 Hydroxypropyldimonium Chloride | 4.0 |
| Cocamidopropyl Betaine | 5.6 |
| Micro Titamium Dioxide | 0.3 |
| TOTAL | 100.0 |

In another embodiment, the cleansing composition comprises these ingredients:

| | % w/w |
|---|---|
| Sodium Cocoyl Isethionate and Stearic Acid | 59.9 |
| Stearic Acid | 36.7 |
| Aminomethyl Propanol | 3.4 |
| TOTAL | 100.0 |

In yet another embodiment, the cleansing composition comprises a “flexible soap” composition as follows:

| | % w/w |
|---|---|
| Water | 33.59 |
| Ca-Iota Carrageenan | 0.30 |
| Potassium Chloride | 0.70 |
| Sodium Laureth Sulfate | 24.88 |
| Kappa Carrageenan | 1.20 |
| Sodium Cocoyl Isethionate | 1.88 |
| Glycerin | 36.95 |
| Phenoxyethanol | 0.50 |
| TOTAL | 100.00 |

In another embodiment, the cosmetic composition is a moisturizing composition. For example, one moisturizing composition comprises the following ingredients:

| | w/w % |
|---|---|
| Isopropyl Palmitate | 50 |
| Proprietary Polyamide | 15 |
| Cocamidopropyl Betaine | 5 |
| Sodium Lauryl Sulfate | 10 |
| Polyethyleneglycol 400 | 10 |
| Dimethicone | 10 |
| TOTAL | 100 |

Optional ingredients conventionally used in cosmetic compositions may be incorporated into the cosmetic device of this invention. These ingredients include, but are not limited to, perfumes/fragrances, preservatives, colorants, dyes, anti-caking agents, and personal care ingredients, including, but are not limited to, skin and hair care ingredients.

Examples of suitable personal care ingredients useful in the present invention include but are not limited to safe and effective amounts of: humectants, sunscreen actives, skin soothers, anti-irritants, anti-inflammatories, emollients, conditioning agents, moisturizers, deodorants, anti-perspirants, artificial tanning agents, antimicrobial agents, anti-acne agents, anti-wrinkle agents, anti-skin atrophy agents, skin firming agents, anti-itch agents, anti-fungal agents, topical anesthetics, skin tone evening agents, active natural ingredients, agents for minimizing the appearance or retarding regrowth of unwanted hair, skin texture modifiers, and additional cleansing agents.

Emollients function by their ability to remain on the skin surface or in the stratum corneum to act as lubricants, to reduce flaking, and to improve the skin appearance. Typical emollients include fatty esters, fatty alcohols, mineral oil, polyether siloxane copolymers and the like. Examples of suitable emollients include, but are not limited to, polypropylene glycol (“PPG”)-15 stearyl ether, PPG-10 cetyl ether, steareth-10, oleth-8, PPG-4 lauryl ether, vitamin E acetate, PEG-7 glyceryl cocoate, lanolin, and combinations thereof. Vitamin E acetate, PEG-7 glyceryl cocoate and combinations thereof are preferred.

Examples of suitable humectants include polyhydric alcohols. Suitable polyhydric alcohols include, but are not limited to, glycerol (also known as glycerin), polyalkylene glycols, alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-dibutylene glycol, 1,2,6,-hexanetriol, ethoxylated glycerol, propoxylated glycerol and mixtures thereof.

Suitable skin soothers include, but are not limited to, panthenol, bisabolol, allantoin, aloe, and combinations thereof.

Suitable conditioning agents include, but are not limited to, dimethicone propyl PG-betaine, dimethicone copolyols, polyquaternium-10, guar, guar derivatives, and combinations thereof. Suitable anti-acne active ingredients include, but are not limited to, salicylic acid, sulfur, lactic acid, glycolic acid, pyruvic acid, urea, resorcinol, N-acetylcysteine, retinoic acid, benzoyl peroxide, octopirox, triclosan, azelaic acid, phenoxyethanol, phenoxypropanol, flavinoids, derivatives thereof, and combinations thereof. Salicylic acid and benzoyl peroxide are preferred.

The optional ingredients may be incorporated into the cleansing composition by means known in the art. Alternatively, the optional ingredients may be incorporated into or coated onto the matrix by means known in the art, provided that the optional ingredients sufficiently adhere to the matrix until incorporated into the cosmetic device. As used herein “coated” means surface coating and/or at least partially impregnating the matrix. The optional ingredients may be incorporated into or coated onto the matrix or encapsulated into other components such as the binder used to make the matrix by means known in the art, for example, by treatment with an appropriate solution, suspension or slurry of the ingredient in an appropriate liquid, followed by drying by conventional means. See, for example, U.S. Pat. Nos. 4,335,185; 6,376,072; and 6,420,047, the disclosures of which are hereby incorporated by reference.

Discrete Elements

The discrete elements are initially formed into a releasably connected matrix such as nonwoven web. As used herein, the term “releasably connected” shall mean joined but capable of separation upon contact with the cosmetic composition. For example, the discrete elements can be randomly disposed and interlaced fibers releasably bonded together at points where they cross and contact each other. This ultimately allows for a controlled distribution of the discrete elements throughout the cosmetic device. By using a releasably connected matrix, problems such as clumping, sinking or floating of the discrete elements, damage, and inexact proportions may be avoided.

In one embodiment, the discrete elements may be joined together by a binder that is soluble in the cosmetic composition. For example, the binder may be the same or a different cosmetic composition, which upon processing allows the discrete elements to disconnect. Alternatively, water-soluble binders such as Vinamul Resyn 1072 (commercially available from Celanese Emulsions) or water-soluble adhesives may also be used as the binder for cosmetic compositions that contain water.

The matrix may be made of any synthetic or natural discrete elements comprising fibers, strands, or filaments, etc. The discrete elements may be any of several water-insoluble synthetic fibers, ranging from acetate rayon and cellulose (which are relatively supple when wetted with water) to nylon, polyester and isotactic polypropylene (which are relatively firm). Fibers such as nylon and polyester may be oriented to provide further resilience and strength. The type, diameter and length of the fibers may vary according the desired use. For example, a relatively thick resilient fiber may be used in a cosmetic device for washing the hands while thin and more supple fibers may be used in a cosmetic device used as a bath soap.

Further materials for use as discrete elements include polyamides, such as poly(hexamethylene adipamide), polycaproamide and/or copolymers thereof; polyesters, such as poly(ethylene terephthalate); poly(hexahydro-p-xylylene terephthalate), and/or copolymers; polyolefins, such as polypropylene and polyethylene; polyurethanes, polycarbonates, polyacetals, polyacrylics, vinyl polymers, vinylidene polymers, and the like.

In one embodiment, the releasably connected matrix is a nonwoven web comprising fibers releasably connected by a binder. The nonwoven web is relatively stable prior to being placed in the cosmetic composition. Once the cosmetic composition is added, the binder dissolves and releases the fibers into separate elements. If a nonwoven web having a certain density is used for example, the final distribution of fibers in the cosmetic device should be similar especially if the fluid cosmetic composition later solidifies to form a solid or semi-solid device such as a soap bar. It may therefore be possible to control the dispersion of fibers in the cosmetic device by modifying the geometry of the nonwoven web.

In another embodiment, the discrete elements individually comprise a water soluble core surrounded by an erodible shell as disclosed in commonly assigned, copending U.S. application Ser. No. [atty docket J&J 5167], the contents of which are incorporated herein by reference. As used herein, “water-soluble” means that the cores disperse, disintegrate, or dissolve in water via chemical degradation and/or hydrolysis and/or solvation. As used herein, the term “erodible” means the shells degrade or disintegrate via mechanical, thermal, or chemical means. For instance, the erodible shells may be made of water insoluble or water permeable material.

The matrix preferably has appropriate loft and dimensional stability. For example, in forming a cosmetic device comprising a cleansing composition that is 1 inch thick, the matrix must ultimately retain enough height to extend throughout the cosmetic composition and not collapse before the discrete elements separate.

In another embodiment, the matrix comprises hollow fibers as taught in U.S. Pat. Nos. 3,558,420 and 5,937,874, the entirety of both are hereby incorporated by reference. The hollow fibers may be utilized to contain optional ingredients as described above. In this embodiment, the optional ingredients may be drawn into the fiber by capillary action or through the use of vacuum. The fibers may have walls that fracture upon use of the cosmetic device, thereby releasing the optional ingredient(s).

In one embodiment, the discrete elements penetrate the surface of the cosmetic device. When the device is used for cleansing, one or more surfaces may be used to exfoliate the outer layer of the skin. The ability to rub the skin may, for example, replace the need for a washcloth, loofa, puff, or other implement typically used by consumers to clean and stimulate their skin in the bath/shower. The cosmetic device of the present invention provides a solution to common problems associated with many types of bars including bath soaps. It provides an efficient tool for exfoliation and can improve the handling of a bar of soap.

The cosmetic device may be made by any of the conventional methods known in the art. These methods include but are not limited to hot pour and extrusion methods, the particulars of which are known by those skilled in the soap art. If the cosmetic device is a liquid, for example a liquid soap, additional processing may be required.

In one embodiment, the cosmetic device may be prepared by heating a cosmetic composition to a temperature at which it flows, i.e., above its melting point (typically about 70° C. to about 130° C.). A nonwoven web, such as a high loft material comprising fibers releasably connected by a soluble binder, is placed into a mold, such as one made of plastic or rubber. The cosmetic composition is added into the mold with the high-loft material. The cosmetic composition saturates at least a portion of the nonwoven web, the binder dissolves, and the fibers separate. The cosmetic composition is allowed to cool and harden into a solid or semi-solid state. The cosmetic device is de-molded and optionally cut into appropriate shapes and sizes. The process may require larger molds.

Optional ingredients like perfume, skin care ingredients, colorants and sensates may be added.

### EXAMPLE

A cosmetic device, namely a cleansing bar, was made according to the invention as follows. Bricks of Life of the Party™ Moisturizing Clear Glycerin Soap were cut into small cubes. The cubes were placed into a glass container and melted using a microwave, thus rendering the soap composition fluid and flowable. One piece of high-loft fibrous material (Carlee Corporation) having a thickness of about 1 inch was placed into an empty 100 g soap mold. The high-loft material comprised of 6-denier polyester fibers that were releasably connected into a matrix by a water soluble binder (Vinamul Resyn 1072), with the binder accounting for about 20 weight % of the matrix. The molten soap was poured into the mold to fill it to the top, thus saturating the high-loft matrix and allowing the discrete elements to separate within the soap composition. The soap was allowed to cool and solidify to form a cleansing bar.

We claim:

1. A method of making a cosmetic device comprising a cosmetic composition and a plurality of discrete elements, said method comprising the steps of:
a) contacting a releasably connected matrix of discrete elements with a flowable cosmetic composition, such that the cosmetic composition saturates at least a portion of the matrix; and
b) allowing the discrete elements to separate within the cosmetic composition.

2. The method of claim 1, wherein the cosmetic device is a solid.

3. The method of claim 1, wherein the cosmetic device is a liquid.

4. The method of claim 1, wherein the cosmetic device is a semi-solid.

5. The method of claim 1, wherein the cosmetic composition is a cleansing composition.

6. The method of claim 1, wherein the cosmetic composition is a moisturizing composition.

7. The method of claim 1, wherein the discrete elements are releasably connected using a binder.

8. The method of claim 7, wherein the binder is a cosmetic composition.

9. The method of claim 1, wherein the discrete elements comprise fibers selected from polyester, polyolefin, polyamide, rayon, cotton, hemp, wool, nylon, and combinations thereof.

10. The method of claim 1, wherein the discrete elements are water-soluble.

11. The method of claim 10, wherein the discrete elements comprise fibers selected from polyethylene oxide fibers, polyethylene oxide-propylene blend fibers, polylactic acid fibers, polysaccharide fibers, polyvinyl alcohol fibers, and mixtures thereof.

12. The method of claim 1, wherein the discrete elements comprise superabsorbent polymers.

13. The method of claim 1, further comprising molding the cosmetic composition containing the discrete elements.

14. The method of claim 1, further comprising allowing the cosmetic composition containing the separated discrete elements to form a solid or semi-solid.

15. The method of claim 1, wherein the discrete elements are coated with a skin benefit agent, cosmetic agent, or sensate.

16. The method of claim 1, wherein the discrete elements are hollow.
